(19) United States
(12) Patent Application Publication
(10) Pub. No.: US 2026/0256675 A1
(43) Pub. Date: Sep. 3, 2026

(54) DEODORANT COMPOSITION WITH SALICYLIC ACID OR A SALICYLIC ACID DERIVATIVE, ALPHA HYDROXY ACID AND CHITOSAN

(71) Applicant: L'OREAL, Paris (FR)

(72) Inventors: Toni MOINE, Chevilly Larue (FR); Charlotte GOZLAN, Chevilly Larue (FR)

(73) Assignee: L'OREAL, Paris (FR)

(21) Appl. No.: 19/474,275

(22) PCT Filed: Apr. 18, 2024
(86) PCT No.: PCT/EP2024/060532

§ 371 Date: Oct. 10, 2025

(30) Foreign Application Priority Data
Apr. 18, 2023 (EP) 23305593.8

(51) Int. Cl. A61K 8/73; A61K 8/365; A61K 8/368; A61Q 15/00

(52) U.S. Cl. CPC A61K 8/736; A61K 8/365; A61K 8/368; A61Q 15/00

(57) ABSTRACT

The present invention relates to a composition comprising, in a physiologically acceptable medium:
(a) native chitosan,
(b) salicylic acid or one of its derivatives, and
(c) at least one alpha hydroxy acid,
   wherein the content by weight of chitosan is strictly greater than the content by weight of alpha hydroxy acid, and the content by weight of alpha hydroxy acid is strictly greater than the content by weight of salicylic acid or one of its derivatives, the contents by weight being defined with respect to the total weight of the composition.

It also relates to a cosmetic process for preventing and/or treating body odors.

The present invention relates to a deodorant composition with salicylic acid or a salicylic acid derivative, alpha hydroxy acid and chitosan. The present invention also relates to a cosmetic process for preventing and/or treating body odors.

A certain number of human body odors are directly linked with the presence, in excess or not, of body fluids secreted at an epidermis, in particular sweat, and also sebum. Thus, the evolution of body odors is in particular linked with a quantitative and/or qualitative modification of the compounds and/or substance present on the skin surface, also referred to as “skin substrate”, and the degradation of which by the resident skin microflora is the source of the biosynthesis of malodorous volatile compounds. However, for obvious reasons, unpleasant body odors are perceived as socially debilitating and, as such, it seems important to effectively control the biological phenomena generating them.

Sweat is a fluid excreted by two types of sweat glands, namely the eccrine glands and the apocrine glands. Whether of eccrine or apocrine origin, provided that it is not generated excessively, sweat has little or no odor when secreted. It is the contact of sweat and more particularly of apocrine sweat with bacteria, which causes the formation of malodorous volatile compounds, via the bacterial degradation of this sweat. The Corynebacterium xerosis bacterium is in particular known as one of the main bacteria causing unpleasant body odors.

Like sweat, sebum is also capable of undergoing bacterial degradation, and may also generate unpleasant odors.

To combat unpleasant odors, and in particular those linked with perspiration, various compounds have already been proposed which, by topical application, are capable of inhibiting sweating, inhibiting the bacteria responsible for the chemical changes described above, masking said odors, or reducing/eliminating the indigenous microflora. In particular, it is known to use compositions comprising salicylic acid or a salicylic acid derivative to eliminate the Corynebacterium xerosis bacterium in particular.

However, the effectiveness of these compositions is not complete, and furthermore exhibits a limit over time.

There is therefore a need for novel compositions exhibiting an enhanced effectiveness for reducing body odors, in particular caused by bacterial degradation of sweat and/or sebum.

There is also a need for novel compositions exhibiting an effectiveness for reducing body odors, in particular caused by bacterial degradation of sweat and/or sebum, which is longer lasting.

Moreover, the formulation of environmentally friendly cosmetic products, i.e., the design and development of which account for environmental concerns, is becoming a major concern to help meet global challenges.

It has therefore become essential to provide more sustainable compositions and/or preparation processes and/or ingredients thus making it possible to address these environmental concerns.

In this context, it is important to develop novel cosmetic compositions with a better carbon footprint in particular by promoting the use of raw materials that are renewable and/or have a good wilderness quality index and/or are of natural origin and more particularly of plant origin while reducing the use of compounds of petrochemical origin.

The present invention therefore relates to a composition, preferably a cosmetic, more preferably a deodorant, composition, comprising, in a physiologically acceptable medium:
(a) native chitosan,
(b) salicylic acid or one of its derivatives, and
(c) at least one alpha hydroxy acid,
wherein the content by weight of chitosan is strictly greater than the content by weight, preferably the total content by weight, of salicylic acid or one of its derivatives, and the content by weight, preferably the total content by weight, of salicylic acid or one of its derivatives is strictly greater than the content by weight, preferably the total content by weight, of alpha hydroxy acid, the contents by weight being defined with respect to the total weight of the composition.

The inventors surprisingly discovered that adding chitosan and alpha hydroxy acid (AHA) in a composition comprising salicylic acid or one of its derivatives intensifies the antibacterial activity of salicylic acid and its derivatives. In particular, the antibacterial activity of salicylic acid and its derivatives against Corynebacterium xerosis is intensified, and demonstrates a synergistic action between the chitosan/AHA pair and salicylic acid and its derivatives.

“Physiologically acceptable” refers to a medium compatible with keratin materials.

The present invention also relates to the cosmetic use of a composition comprising, in a physiologically acceptable medium:
(a) native chitosan,
(b) salicylic acid or one of its derivatives, and
(c) at least one alpha hydroxy acid,
for preventing and/or reducing body odors.

Preferably, the cosmetic use according to the invention is for reducing body odors at the underarm regions, peri-anal areas, feet and/or breast areolae.

Preferably, native chitosan and/or salicylic acid or one of its derivatives and/or alpha hydroxy acid has/have one or more features as defined in the description above.

Preferably, the composition of the cosmetic use is a composition according to the invention.

The present invention also relates to a cosmetic process for preventing and/or reducing body odors, comprising contacting a keratin material, preferably the skin, with a composition comprising, in a physiologically acceptable aqueous medium:
(a) native chitosan,
(b) salicylic acid or one of its derivatives, and
(c) at least one alpha hydroxy acid.

Preferably, the composition according to the invention is applied on the skin at the underarm regions, peri-anal areas, feet and/or breast areolae.

Preferably, native chitosan and/or salicylic acid or one of its derivatives and/or alpha hydroxy acid has/have one or more features as defined in the description above.

Preferably, the composition of the cosmetic process is a composition according to the invention.

Preferably, the composition according to the invention is substantially free from (meth)acrylic polymer. Preferably, the composition according to the invention is substantially free from silicone.

“Substantially free from (meth)acrylic polymer” means that the composition comprises less than 1% by weight with respect to the total weight of the composition, preferably less than 0.5% by weight, preferably less than 0.3% by weight, preferably 0.1% by weight of (meth)acrylic polymer. Preferably, the composition is totally free from (meth)acrylic polymer. The term (meth)acrylic polymer denotes a polymer comprising at least one acrylic acid monomer or at least one methacrylic acid monomer.

“Substantially free from silicone” means that the composition comprises less than 1% by weight with respect to the total weight of the composition, preferably less than 0.5% by weight, preferably less than 0.3% by weight, preferably 0.1% by weight of silicone. Preferably, the composition is totally free from silicone. Silicone refers to any silicone compound.

### Chitosan

The composition according to the invention comprises at least one native chitosan.

Preferably, the native chitosan has a molecular weight strictly greater than 3000 Dalton (3 kDa; 1 Da=1 g/mol), preferably greater than or equal to 10 kDa, preferably greater than or equal to 15 kDa, preferably greater than or equal to 20 kDa. Preferably, the native chitosan has a molecular weight between 10 kDa and 2 MDa, preferably between 15 kDa and 1.5 MDa, preferably between 20 kDa and 300 kDa, preferably between 20 kDa and 200 kDa.

Chitosan is very scarce in nature. It is only reported in the exoskeletons of certain insects such as termite queens and in the cell walls of a particular class of fungi, zygomycetes.

Chitosan is obtained by deacetylating chitin. Chitin is a polysaccharide composed of several N-acetyl-D-glucosamine units interlinked by a β (1,4) type bond.

The ideal chemical structure of chitosan is a chain of β-D-glucosamine monomers linked by a glycoside (1->4) bond.

According to the invention, “chitosan” refers to any copolymer formed from N-acetyl-D-glucosamine and D-glucosamine constituent units, the degree of acetylation of which is less than 90%. Chitosan consists of glucosamine sugar units (deacetylated units) and N-acetyl-D-glucosamine units (acetylated units) interlinked by β (1,4) type bonds and represents a Poly(N-acetyl-D-glucosamine)-poly(D-glucosamine) type polymer.

Preferably, the degree of acetylation of the chitosan is less than or equal to 80%, preferably less than or equal to 70%, preferably less than or equal to 60%, preferably less than or equal to 50%, preferably less than or equal to 35%, preferably less than or equal to 25%, preferably less than or equal to 15%, preferably between 1% and 50%.

The degree of acetylation is the percentage of acetylated units relative to the number of total units, it can be determined by Fourier-transform infrared (TRIF) spectroscopy or by titration with a strong base.

The chitosan according to the invention is preferably a polysaccharide prepared from a fungal source. It is in particular extracted and purified from safe and plentiful dietary or biotechnological fungal sources such as Agaricus bisporus or Aspergillus niger.

The chitosan according to the invention is preferably obtained from the mycelium of an Ascomycetes type fungus, and in particular of Aspergillus niger and/or a Basidiomycetes fungus, and in particular Lentinula edodes (shiitake) and/or Agaricus bisporus. Preferably, the fungus is Aspergillus niger.

The chitosan may be of GMO origin, but preferably is of non-GMO origin.

The chitosan according to the invention is native, i.e., it is not modified. It in particular contains no chemical modification.

A method for preparing chitosan is that described in the application WO03068824.

Preferably, the chitosan used in the invention is in powder form. It is in particular marketed by Kitozyme under the name Kiosmetine or Kionutrime.

Chitosan is preferably present at a quantity ranging from 0.1% to 10% by weight, preferably from 0.2% to 5% by weight, preferably from 0.3% to 3% by weight, preferably from 0.5% to 2% by weight, preferably from 0.8% to 1.6% by weight with respect to the total weight of the composition.

### Salicylic Acid or One of its Derivatives

The composition according to the invention comprises salicylic acid or one of its derivatives.

The salicylic acid derivatives according to the invention are preferably lipophilic derivatives. Lipophilic salicylic acid derivative refers to any salicylic acid derivative non-soluble at 1% by weight in water and at 25° C.

The salicylic acid derivatives are preferably according to the following formula (I):

### [Chem 1]

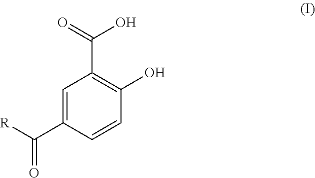

wherein R denotes a saturated, linear, branched or cyclic aliphatic chain, having from 2 to 22 carbon atoms; an unsaturated, linear or branched chain, having from 2 to 22 carbon atoms containing one or more double bonds that can be conjugated; an aromatic ring bonded to the carbonyl radical directly or via saturated or unsaturated aliphatic chains having from 2 to 8 carbon atoms; said groups being capable of being substituted by one or more substituents, identical or different, chosen from (A) halogen atoms (B) the trifluoromethyl group, (C) hydroxyl groups in free or esterified form by an acid having from 1 to 6 carbon atoms or (D) a carboxyl function in free form or esterified by a lower alcohol having from 1 to 6 carbon atoms, as well as their salts obtained by salification with a mineral or organic base.

Preferably, R denotes a saturated, linear, branched or cyclic aliphatic chain containing from 3 to 11 carbon atoms; an unsaturated, linear or branched chain, containing from 3 to 17 carbon atoms and comprising one or more optionally conjugated double bonds; said hydrocarbon groups being capable of being substituted by one or more substituents, identical or different, chosen from (A) halogen atoms (B) the trifluoromethyl group, (C) hydroxyl groups in free or esterified form by an acid having from 1 to 6 carbon atoms or (D) a carboxyl function in free form or esterified by a lower alcohol having from 1 to 6 carbon atoms;

as well as their salts obtained by salification with a mineral or organic base.

Preferably, R is a linear or branched C3-C11 alkyl group.

Among the particularly preferred compounds of formula (I), mention may be made of n-octanoyl-5-salicylic acid (capryloyl salicylic acid); n-decanoyl-5-salicylic acid; n-dodecanoyl-5-salicylic acid; n-heptyloxy-5-salicylic acid and their corresponding salts.

Preferably, the composition according to the invention comprises salicylic acid or n-octanoyl-5-salicylic acid, advantageously comprises N-octanoyl-5-salicylic acid.

The salts of the compounds of formula (I) may be obtained by salification with a mineral or organic base. By way of example of a mineral base, mention may be made of alkali or alkaline-earth metal hydroxides such as sodium hydroxide, potassium hydroxide or ammonia.

Among the organic bases, mention may be made of amines and alkanolamines.

Quaternary salts such as those described in the patent FR 2.607.498 are of particular interest.

Preferably, the composition according to the invention comprises, with respect to the total weight of the composition, from 0.01% to 5% by weight, preferably from 0.02% to 3% by weight, preferably from 0.05% to 2% by weight, preferably from 0.1% to 1% by weight, preferably from 0.15% to 0.5% by weight, of salicylic acid or one of its derivatives.

Preferably, the weight ratio between the content by weight of chitosan and the content by weight of salicylic acid or one of its derivatives ranges from 1.1 to 10, preferably from 2 to 8, advantageously from 4 to 6.

### Alpha Hydroxy Acid (AHA)

The composition according to the present invention comprises at least one alpha hydroxy acid (AHA).

Alpha hydroxy acid refers according to the invention to a carboxylic acid having at least one hydroxy function occupying an alpha position on said acid (carbon adjacent to a carboxylic acid function). This acid may be presented in the final composition in the form of free acid and/or in the form of one of its associated salts (salts with an organic base or an alkali in particular), in particular according to the final pH imposed on the composition.

The alpha hydroxy acids are for example chosen from lactic acid, citric acid, methyllactic acid, glucuronic acid, glycolic acid, pyruvic acid, 2-hydroxy-butanoic acid, 2-hydroxypentanoic acid, 2-hydroxyhexanoic acid, 2-hydroxyheptanoic acid, 2-hydroxyoctanoic acid, 2-hydroxy-nonanoic acid, 2-hydroxydecanoic acid, 2-hydroxyundecanoic acid, 2-hydroxydodecanoic acid, 2-hydroxytetradecanoic acid, 2-hydroxy-hexadecanoic acid, 2-hydroxyoctadecanoic acid, 2-hydroxytetra-cosanoic acid, 2-hydroxyeicosanoic acid; mandelic acid; phenyllactic acid; gluconic acid; galacturonic acid; aleuritic acid; ribonic acid; tartronic acid; tartaric acid; malic acid; fumaric acid; their salts and their mixtures.

According to a preferred embodiment, the alpha hydroxy acid is chosen from lactic acid, gluconic acid, citric acid, malic acid, glycolic acid, tartaric acids and their salts. More particularly, the alpha hydroxy acid is chosen from lactic acid, citric acid, their salts and mixtures thereof.

Advantageously, the alpha hydroxy acid is lactic acid, its salts and mixtures thereof.

The alpha hydroxy acid(s) may be present at a quantity ranging for example from 0.05% to 5% by weight, preferably from 0.1% to 3% by weight, preferably from 0.2% to 1% by weight, preferably from 0.3% to 0.8% by weight with respect to the total weight of the composition.

Preferably, the weight ratio between the content by weight of chitosan and the content by weight, preferably the total content by weight, of alpha hydroxy acid(s) ranges from 1.1 to 5, preferably from 1.2 to 4, advantageously from 1.5 to 2.5.

Such a ratio makes it possible advantageously to improve the solubilization of chitosan, and therefore obtain the booster effect of salicylic acid and its derivatives.

Preferably, such a ratio also allows formulating a composition with a pH value equal to or greater than 3.5, which is the minimum pH value that is physiologically acceptable for the axillary zone. This therefore avoids a drastic reduction in axillary pH that may be caused by the application of the deodorant composition.

Preferably, the weight ratio between the content by weight, preferably the total content by weight, of alpha hydroxy acid(s) and the content by weight, preferably the total content by weight, of salicylic acid or one of its derivatives ranges from 1.1 to 5, preferably from 1.2 to 4, advantageously from 1.5 to 3.

### Polyols

The composition according to the present invention may also comprise at least one polyol.

The term “polyol” refers here to an alcohol having two or more hydroxy groups and does not include saccharide or derivatives thereof. The derivative of a saccharide includes a sugar alcohol obtained by reducing one or more carbonyl groups of a saccharide, as well as a saccharide or a sugar alcohol wherein the hydrogen atom(s) in one or more hydroxy groups thereof has/have been replaced by at least one substituent such as an alkyl group, a hydroxyalkyl group, an alkoxy group, an acyl group or a carbonyl group.

The polyol may be a C2-24 polyol, preferably a C2-9 polyol, comprising at least 2 hydroxy groups, and preferably 2 to 5 hydroxy groups.

The polyol may be a natural or synthetic polyol. The polyol may have a linear, branched or cyclic molecular structure.

The polyol may be chosen from glycerins and their derivatives, as well as glycols and their derivatives. The polyol may be chosen in the group consisting of glycerin, diglycerin, polyglycerin, ethyleneglycol, diethyleneglycol, propyleneglycol, dipropyleneglycol, butyleneglycol, pentyleneglycol, hexyleneglycol, 1,3-propanediol, 1,4-butanediol, 1,5-pentanediol, and polyethyleneglycol, in particular having from 5 to 50 ethylene oxide groups and mixtures thereof; more particularly chosen from glycerin, propyleneglycol, dipropyleneglycol, butyleneglycol and mixtures thereof.

The polyol is preferably polyethylene glycol.

The polyol may be present in the composition according to the invention at a content ranging from 1% to 50% by weight, with respect to the total weight of the composition, preferably ranging from 5% to 40% by weight, preferably ranging from 8% to 30% by weight, preferably ranging from 10% to 20% by weight.

### pH of the Composition

Preferably, the composition according to the invention has a pH less than or equal to 7, preferably less than or equal to 6.5, preferably less than or equal to 6.3. Advantageously, the pH of the composition is between 3 and 6.3, preferably between 3.5 and 6.3, preferably between 4 and 6.3.

### Physiologically Acceptable Aqueous Medium

The physiologically acceptable aqueous medium comprises water.

The water used may be sterile demineralized water and/or floral water such as rose water, cornflower water, chamomile water or linden water, and/or a spring or natural mineral water, such as for example: Vittel water, water from the basin of Vichy, Uriage water, la Roche Posay water, la Bourboule water, Enghien-les-bains water, Saint Gervais-les-Bains water, Neris-les-Bains water, Allevar-les-Bains water, Digne water, Maizières water, Neyrac-les-Bains water, Lons-le-saunier water, les Eaux Bonnes, Rochefort water, Saint Christau water, Fumades water and Tercis-les-bains water, Avène water

The first composition preferably comprises at least 50% by weight of water with respect to the total weight of the first composition, preferably at least 60% by weight.

The composition preferably comprises from 50% to 95% by weight of water with respect to the total weight of the composition, more preferably from 60% to 90% by weight.

Moreover, the composition according to the invention may also comprise a non-water-miscible organic phase, known as a fatty phase. This generally comprises one or more hydrophobic compounds rendering said phase non-water-miscible. Said phase may be liquid (in the absence of a structuring agent) at ambient temperature (20-25° C.). Preferentially, the non-water-miscible organic liquid phase according to the invention generally comprises at least one volatile or non-volatile oil, and optionally at least one structuring agent. “Oil” refers to a fat that is liquid at ambient temperature (25° C.) and atmospheric pressure (760 mm Hg, namely 105 Pa). The oil may be volatile or non-volatile.

The composition according to the invention may also comprise at least one solid fat. Preferably, this solid fat is chosen from waxes and pasty fats and mixtures thereof.

The composition according to the invention may also comprise at least one additional deodorant active agent, different from the native chitosan, salicylic acid or one of its derivatives and alpha hydroxy acid of the composition according to the invention.

The term “deodorant active agent” refers in the context of the present invention to any active agent, alone, with the effect of masking, absorbing, improving and/or reducing the unpleasant odor resulting from the decomposition of human sweat.

By way of illustration of these additional deodorant active agents, mention may in particular be made of zinc neodecanoate, the zinc salt of L-Pyrrolidone Carboxylic acid (zinc PCA), zinc gluconate or octenidine hydrochloride.

The additional deodorant active agents may be present preferably in the compositions according to the invention in weight concentrations ranging from 0.01 to 10% by weight with respect to the total weight of the composition.

The composition according to the invention may further comprise cosmetic adjuvants chosen from opacifiers, stabilizers, preservatives, perfumes, cosmetic active agents, fillers, suspension agents, sequestering agents, dyestuffs or any other ingredient routinely used in cosmetics for this type of application. Obviously, a person skilled in the art will take care to choose this or these optional additional compound(s) in such a way that the advantageous properties of the composition according to the invention are not altered, or are not substantially altered, by the envisaged addition(s).

### Dosage Forms

The composition according to the invention may independently be presented in any of the dosage forms conventionally used for topical application and particularly in the form of aqueous gels, aqueous or hydroalcoholic solutions. They may, by the addition of a fatty or oil phase, be in the form of dispersions such as lotion, emulsions of liquid or semi-liquid consistency such as milk, obtained by dispersing a fatty phase in an aqueous phase (O/W) or conversely (W/O), or suspensions or emulsions of soft, semi-solid or solid consistency such as cream or gel, or multiple emulsions (W/O/W or O/W/O), microemulsions, ionic and/or non-ionic type vesicle dispersions, or wax/aqueous phase dispersions. The composition according to the invention may be particularly packaged in pressurized form in an aerosol device or in a pump bottle; packaged in a device equipped with a perforated wall particularly a grid; packaged in a device equipped with a roll-on applicator; packaged in stick form, in loose or compact powder form. In this respect, they contain the ingredients generally used in this type of products and well-known to a person skilled in the art.

The composition according to the invention can be obtained conventionally by those skilled in the art.

The expressions “between . . . and . . . ” and “ranging from . . . to . . . ” are to be understood to be inclusive of the limits, unless specified otherwise.

Concrete, yet non-limiting, examples, illustrating the invention, will now be provided.

### EXAMPLES

### Example 1: Preparation of a Composition According to the Invention and Comparative Compositions

Compositions C1 and C2 according to the invention and comparative compositions C3* and C5* from Table 1 hereinafter are prepared using the following protocol:
Preparation of phase A by dispersing chitosan in water, then adding lactic acid (pH=5),
Preparation of phase B by solubilizing capryloyl salicylic acid in propylene glycol,
Introduction of phase B in phase A under stirring until a surface-foaming brown liquid of pH=4.6 is obtained.

For composition C4*, phase A is prepared by preparing xanthan gel by mixing the two gums in water. Phase B is prepared by solubilizing capryloyl salicylic acid with propylene glycol. Then phase B is introduced into phase A and caprylyl glycol is added.

TABLE 1

| Ingredient (% w/w) | C1 | C2 | C3* | C4* | C5* |
|---|---|---|---|---|---|
| Water | QS 100 | QS 100 | QS 100 | QS 100 | QS 100 |
| Propylene glycol | 15 | 15 | 15 | 15 | — |
| Chitosan (27 kDa) | 1 | — | 1 | — | 0.1 |
| Chitosan (110 kDa) | — | 1 | — | — | — |
| Capryloyl salicylic | 0.2 | 0.2 | — | 0.2 | — |
| acid | | | | | |
| Lactic acid | 0.45 | 0.45 | 0.45 | — | 0.07 |
| Dehydrated xanthan | — | — | — | 0.29 | — |
| gum | | | | | |
| Xanthan gum | — | — | — | 0.39 | — |
| Caprylyl glycol | — | — | — | 0.5 | — |

### Example 2: Evaluation of Antimicrobial Activity of Compositions C1, C2 and C3* to C5*

The antimicrobial activity of compositions C1 and C2 according to the invention, and of comparative compositions C3* to C5* was evaluated using the Corynebacterium xerosis bacterium, according to the following protocol:

In a suitable culture medium, the biocidal effect of the formulas is evaluated on strains of interest after 2, 6, and 24 hours of contact versus a placebo (i.e., medium without formula). The logarithmic reduction of the number of viable organisms is measured at each contact time.

The “underarm odor” model strain is Corynebacterium freneyi CIP 52.16 (ex Corynebacterium xerosis).

This strain is brought into contact with the formula being tested in a culture medium, in the following proportions:
10% of the inoculum at 10⁸ microorganisms/ml,
10% of the formula being tested, and
80% culture medium (Trypcase Soy broth).

At the same time, a growth control, in which the formula is replaced with a diluent, is prepared under the same conditions.

The samples are placed in an incubator at a temperature of 35° C. (optimum growth temperature for the strain) with continuous stirring during the test.

After 2, 6, and 24 hours of contact, the number of viable microorganisms remaining in the mixture is evaluated.

The results are expressed as a logarithm of the number of microorganisms per milliliter of mixture.

The result obtained with the formula being tested is compared to the results of the growth control, and the difference is expressed as a Log number difference at T equals 24 hours.

The results obtained are presented in Table 2 hereinafter, indicating the number of viable Corynebacterium xerosis bacteria as a function of time, and the composition used (the detection threshold being 2.3).

TABLE 2

Time (hours)

| Composition | 0 | 2 | 6 | 24 |
|---|---|---|---|---|
| Control | 7.6 | 7.6 | 7.7 | 8.4 |
| C1 | 7.4 | 4.3 | 3.7 | 1.6 |
| C2 | 7.4 | 4.1 | 4.0 | 1.7 |
| C3* | 7.4 | 4.1 | 3.9 | 3.0 |
| C4* | 7.4 | 6.9 | 6.7 | 5.9 |
| C5* | 7.1 | 4.1 | 2.8 | 5.6 |

The antimicrobial activity of comparative composition C4* comprising only capryloyl salicylic acid and comparative composition C3* comprising only chitosan decreases over time, in particular between 6 and 24 hours. On the other hand, composition C1 according to the invention comprising the combination of chitosan and capryloyl salicylic acid shows an excellent antimicrobial activity, even after 24 hours. Indeed, the number of viable bacteria being below the detection threshold, this means that, after 24 hours, all the bacteria are eliminated by composition C1 according to the invention.

These results therefore show that adding chitosan in a composition comprising capryloyl salicylic acid makes it possible to boost its antimicrobial activity.

### Example 3: Evaluation of Chemical and Physical Properties of Compositions

Compositions C6 and C7 according to the invention and comparative compositions C8* and C9* are prepared following a similar protocol for preparing compositions C1 and C2 as disclosed in Example 1.

In Table 3 below, for each composition C6, C7, C8* and C9*, the content by weight of each ingredient with respect to the total weight of the composition is listed.

Compositions of the invention C6 and C7 present a weight ratio chitosan:alpha hydroxy acid (lactic acid) of around 2.22.

Comparative compositions C8* and C9* present a weight ratio chitosan:alpha hydroxy acid (lactic acid) of 1, i.e. the weight content of chitosan is equal to the weight content of lactic acid.

TABLE 3

| Ingredient (% w/w) | C6 | C7 | C8* | C9* |
|---|---|---|---|---|
| Water | QS 100 | QS 100 | QS 100 | QS 100 |
| Propylene glycol | 15 | 15 | 15 | 15 |
| Chitosan (27 kDa or | 1 | 1 | 0.5 | 1 |
| 110 kDa) | | | | |
| Salicylic acid | — | 0.2 | — | — |
| Capryloyl salicylic acid | 0.2 | — | 0.2 | 0.2 |
| Lactic acid | 0.45 | 0.45 | 0.5 | 1 |

After preparation, the pH of each composition is measured, and the appearance of these compositions is observed and described in Table 4:

TABLE 4

| Composition | C6 | C7 | C8* | C9* |
|---|---|---|---|---|
| pH | 4.6 | 3.7 | 3.3 | 3.6 |
| Appearance | Cloudy, | Translucent, | Cloudy, non- | Cloudy, non- |
| | redispersible | well-dispersed | dispersible | dispersible |

The results show that only compositions C6 and C7 according to the invention exhibit a good dispersion: the cloudy C6 composition can be easily redispersed, and C7 exhibits a well-dispersed translucent appearance.

In contrast, both of the comparative compositions C8* and C9* are cloudy, and further present white volutes which are impossible to be redispersed.

In addition, compositions C6 and C7 according to the invention exhibit higher pH values than the ones of comparative compositions C8* and C9*.

1. A composition, comprising in a physiologically acceptable aqueous medium:
(a) native chitosan,
(b) salicylic acid or one of its derivatives, and
(c) at least one alpha hydroxy acid,
wherein the content by weight of chitosan is strictly greater than the content by weight of alpha hydroxy acid, and the content by weight of alpha hydroxy acid is strictly greater than the content by weight of salicylic acid or one of its derivatives, the contents by weight being defined with respect to the total weight of the composition.

2. The composition according to claim 1, comprising, with respect to the total weight of composition, from 0.1% to 10% by weight of native chitosan.

3. The composition according to claim 1, wherein the native chitosan (a) has a molecular weight strictly greater than 3000 Dalton and/or the degree of acetylation of the chitosan is less than or equal to 50%.

4. The composition according to claim 1, wherein the chitosan (a) is extracted and purified from dietary or biotechnological fungal sources.

5. The composition according to claim 1, comprising, with respect to the total weight of composition, from 0.01% to 5% by weight of salicylic acid or one of its derivatives.

6. The composition according to claim 1, comprising a salicylic acid derivative of formula (I): 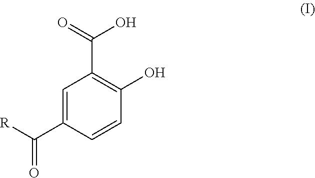
wherein R denotes a saturated, linear, branched or cyclic aliphatic chain, having from 2 to 22 carbon atoms; an unsaturated, linear or branched chain, having from 2 to 22 carbon atoms containing one or more double bonds that can be conjugated; an aromatic ring bonded to the carbonyl radical directly or via saturated or unsaturated aliphatic chains having from 2 to 8 carbon atoms; said groups being capable of being substituted by one or more substituents, identical or different, chosen from (A) halogen atoms (B) the trifluoromethyl group, (C) hydroxyl groups in free or esterified form by an acid having from 1 to 6 carbon atoms or (D) a carboxyl function in free form or esterified by a lower alcohol having from 1 to 6 carbon atoms,
as well as their salts obtained by salification with a mineral or organic base,
preferably, the salicylic acid derivative of formula (I) is chosen from n-octanoyl-5-salicylic acid (or capryloyl salicylic acid); n-decanoyl-5-salicylic acid; n-dodecanoyl-5-salicylic acid; n-heptyloxy-5-salicylic acid and their corresponding salts.

7. The composition according to claim 1, wherein the alpha hydroxy acid is chosen from lactic acid, gluconic acid, citric acid, malic acid, glycolic acid, tartaric acids and their salts.

8. The composition according to claim 1, comprising, with respect to the total weight of composition, from 0.05% to 5% by weight of alpha hydroxy acid.

9. The composition according to claim 1, which has a pH less than or equal to 7.

10. The composition according to claim 1, further comprising at least one polyol at a quantity ranging 1 and 50% by weight, with respect to the total weight of the composition.

11. The composition according to claim 1 which is substantially free from (meth)acrylic polymer and/or silicone.

12. (canceled)

13. A cosmetic process for preventing and/or reducing body odors, comprising contacting a keratin material with a composition comprising,
(a) native chitosan,
(b) salicylic acid or one of its derivatives, and
(c) at least one alpha hydroxy acid.

14. The composition according to claim 2, wherein the native chitosan (a) has a molecular weight strictly greater than 3000 Dalton and/or the degree of acetylation of the chitosan is less than or equal to 50%.

15. The composition according to claim 2, wherein the chitosan (a) is extracted and purified from dietary or biotechnological fungal sources.

16. The composition according to claim 3, wherein the chitosan (a) is extracted and purified from dietary or biotechnological fungal sources.

17. The composition according to claim 2, comprising, with respect to the total weight of composition, from 0.01% to 5% by weight of salicylic acid or one of its derivatives.

18. The composition according to claim 3, comprising, with respect to the total weight of composition, from 0.01% to 5% by weight of salicylic acid or one of its derivatives.

19. The composition according to claim 4, comprising, with respect to the total weight of composition, from 0.01% to 5% by weight of salicylic acid or one of its derivatives.

20. The composition according to claim 2, comprising a salicylic acid derivative of formula (I): 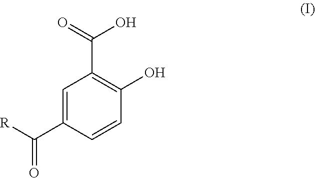
wherein R denotes a saturated, linear, branched or cyclic aliphatic chain, having from 2 to 22 carbon atoms; an unsaturated, linear or branched chain, having from 2 to 22 carbon atoms containing one or more double bonds that can be conjugated; an aromatic ring bonded to the carbonyl radical directly or via saturated or unsaturated aliphatic chains having from 2 to 8 carbon atoms; said groups being capable of being substituted by one or more substituents, identical or different, chosen from (A) halogen atoms (B) the trifluoromethyl group, (C) hydroxyl groups in free or esterified form by an acid having from 1 to 6 carbon atoms or (D) a carboxyl function in free form or esterified by a lower alcohol having from 1 to 6 carbon atoms,
as well as their salts obtained by salification with a mineral or organic base.

21. The composition according to claim 3, comprising a salicylic acid derivative of formula (I): 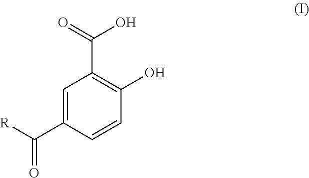
wherein R denotes a saturated, linear, branched or cyclic aliphatic chain, having from 2 to 22 carbon atoms; an unsaturated, linear or branched chain, having from 2 to 22 carbon atoms containing one or more double bonds that can be conjugated; an aromatic ring bonded to the carbonyl radical directly or via saturated or unsaturated aliphatic chains having from 2 to 8 carbon atoms; said groups being capable of being substituted by one or more substituents, identical or different, chosen from (A) halogen atoms (B) the trifluoromethyl group, (C) hydroxyl groups in free or esterified form by an acid having from 1 to 6 carbon atoms or (D) a carboxyl function in free form or esterified by a lower alcohol having from 1 to 6 carbon atoms,
as well as their salts obtained by salification with a mineral or organic base.
